(19) United States
(12) Patent Application Publication
(10) Pub. No.: US 2012/0004528 A1
(43) Pub. Date: Jan. 5, 2012

(54) Implantable Remote Monitoring Sensor

(75) Inventors: Bing Li, Arlington, TX (US); Marsha A. McLaughlin, Joshua, TX (US); Yin Yang, Arlington, TX (US)

(73) Assignee: ALCON RESEARCH, LTD., Fort Worth, TX (US)

(21) Appl. No.: 12/829,968

(22) Filed: Jul. 2, 2010

(51) Int. Cl. A61B 3/16

(52) U.S. Cl. 600/398

(57) ABSTRACT

An exemplary implantable sensor includes a housing that generally conforms to a curvature of an anterior chamber of a patient's eye. A sensing device disposed in the housing is configured to detect physiological parameters and transmit a signal representing the physiological parameters. The exemplary sensor may be used to detect physiological parameters such as intraocular pressure, flow velocity of aqueous humor, blood sugar, and blood biochemicals.

### BACKGROUND

Aqueous humor fills the space between the lens and the cornea of the human eye and is similar to the compositions of blood plasma except aqueous humor has less protein. Intraocular pressure is the fluid pressure of the aqueous humor. Changes in intraocular pressure are a sign of glaucoma. If left undiagnosed and untreated, glaucoma can lead to blindness. Thus, measuring intraocular pressure is an important part of diagnosing and treating eye diseases.

Various techniques may be used to remotely monitor and measure intraocular pressure. For example, a wired sensor may be embedded into a contact lens. However, this technique requires that the lens be molded as an exact copy of the eye surface, and the accuracy of the measurements using this technique are affected by tissue wall thickness, rigidity, eye size, and other physiological parameters such as eye movement and lid pressure. Another technique to measure intraocular pressure is to use wireless sensors built into an intraocular lens, but this technique requires replacing the patient's native lens. A third technique is to implant a pressure transducer subcutaneously on the back of the patient's neck and to measure intraocular pressure via a fluid-filled catheter and needle probe that conducts pressure from the anterior chamber to the transducer. The needle is glued into the anterior chamber. This technique, however, can cause irritation, cornea scarring, inflammation, and limitations of eye movement.

Accordingly, an implantable sensor for monitoring intraocular pressure and other physiological parameters that is easy to manufacture and implant in the patient's eye is needed.

### BRIEF DESCRIPTION OF THE DRAWINGS

FIGS. 1A-1D illustrate exemplary implantable sensors with various configurations of exemplary fixation elements.

FIG. 2 illustrates an exemplary implantable sensor with an antenna disposed on a surface of a housing.

FIG. 3 illustrates an exemplary cross-sectional view of a portion of the implantable sensor of FIG. 1A taken along line 3-3.

FIG. 4 illustrates the exemplary implantable sensor of FIG. 1A disposed in the anterior chamber of a patient's eye.

FIG. 5 illustrates an exemplary system using an embodiment of the implantable sensor.

### DETAILED DESCRIPTION

An exemplary implantable sensor includes a housing that generally conforms to a curvature of an anterior chamber of a patient's eye. A sensing device disposed in the housing is configured to detect physiological parameters and transmit a signal representing the physiological parameters. The shape of the sensor helps anchor the sensor inside the anterior chamber of the patient's eye. As described herein, the sensor may be used to detect intraocular pressure and other physiological parameters. Moreover, the sensor is designed for ease of manufacture and implantation in a patient's eye.

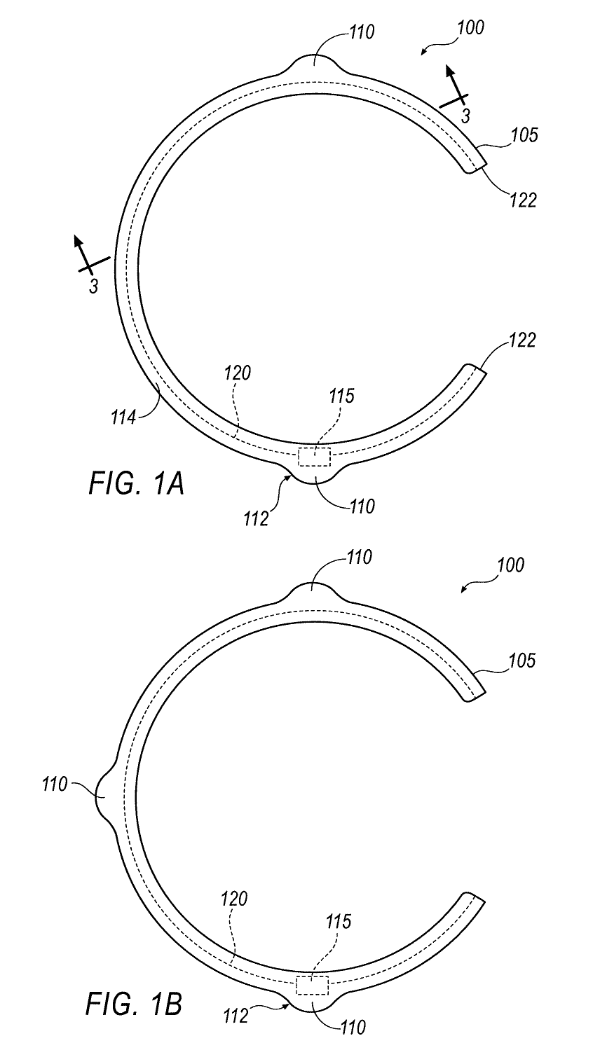

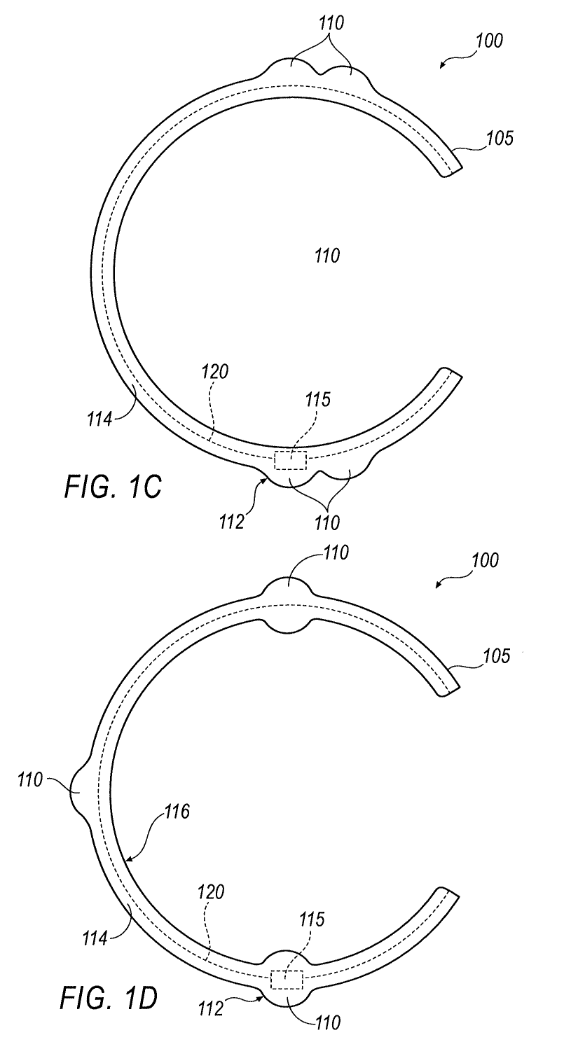

FIGS. 1A-1D illustrate exemplary implantable sensors 100 configured to detect at least one physiological parameter and transmit a signal representing the physiological parameter. The sensor 100 may take many different forms and include multiple and/or alternate components and facilities. While exemplary sensors 100 are shown in the accompanying figures, the exemplary components illustrated are not intended to be limiting. Indeed, additional or alternative components and/or implementations may be used.

The sensor 100 includes a housing 105, one or more fixation elements 110, a sensing device 115, and an antenna 120. In general, the housing 105 supports the sensing device 115 and antenna 120 inside a patient's eye, and the fixation elements 110 limit movement of the sensor 100 within the patient's eye. The housing 105 has a configuration that generally conforms to a curvature of an anterior chamber of the patient's eye. For example, the housing 105 may have a generally C-shaped configuration that may be formed by extending two ends 122 of the housing 105 toward one another and leaving an opening between the two ends 122. With this shape, the housing 105 allows light to reach the patient's retina (or eye fundus) and does not expose the patient to a significant risk of damage to the iris and the corneal endothelium. The housing 105 may have other configurations such as a substantially G-shaped or O-shaped configuration. Moreover, although illustrated as having a substantially curved periphery 112 in FIGS. 1A-1D, the housing 105 may have corners. For instance, the housing 105 may also be configured with arms (not shown) that extend at angles relative to one another.

The housing 105 may be formed to any size that fits within the anterior chamber of the patient's eye. For one exemplary patient, the depth of the anterior chamber may be between approximately 2.9-3.3 mm and the width of the anterior chamber may about 12.53±0.47 mm. However, the size of the anterior chamber may change for each patient. Accordingly, the housing 105 may have an overall length that is approximately two-thirds of the anterior chamber circumference length or a diameter in the range of 11.0-14.0 mm. An opening between the two ends 122 of the housing 105 is approximately 2-6 mm wide. Of course these sizes are merely exemplary. The size of the housing 105 may be customized to fit within a specific patient's eye, or alternatively, the housing 105 may be sized based on one or more demographics. For example, the housing 105 of a sensor 100 designed for an adult patient may be larger than the housing 105 of a sensor 100 designed for a child patient.

The fixation elements 110 are configured to extend from an outer periphery 112 of the housing 105 and anchor the housing 105 inside the patient's eye in a way that allows the sensor 100 to measure one or more physiological parameters. Instead of extending from the outer periphery 112, the fixation elements 110 may additionally or alternatively extend from a top or bottom surface 114 of the housing 105, or both. Moreover, the fixation elements 110 may be flush with the top and/or bottom surfaces 114. In one exemplary approach, the fixation elements 110 may be the only points of contact between the sensor 100 and, for instance, the patient's corneal endothelium.

The fixation element 110 may be integrally formed with the housing 105 or attached to the housing 105. For example, the fixation elements 110 may be semi-circular protrusions integrally formed with the housing 105. As illustrated in FIG. 1A, the sensor 100 includes two fixation elements 110 defined by protrusions extending outwardly from an outer periphery 112 of the housing 105. In one exemplary arrangement, the two fixation elements are disposed on opposite sides of the housing 105 in an opposing manner. However, the sensor 100 may include any number of fixation elements 110 at various locations and orientations. For instance, FIG. 1B illustrates the sensor 100 having three fixation elements 110, two of which are located across from one another and the third is located opposite the opening defined between the two ends 122 of the housing 105. FIG. 1C illustrates two groups of fixation elements 110, each group having two fixation elements 110 disposed adjacent to each other. FIG. 1D illustrates the sensor 100 having two fixation elements 110 opposing one another such that one fixation element 110 extends from the outer periphery 112 of the housing 105 and the other extends from an inner periphery 116 of the housing 105. In the exemplary configuration shown in FIG. 1D, the opposing arrangement of fixation elements 110 gives the appearance of a circular fixation element 115. Of course, the fixation elements 110 may have any shape (e.g., circular, semi-circular, triangular, square, etc.) and the sensor 100 may have multiple fixation elements 110, one or more of which have different shapes.

The sensing device 115 is configured to detect physiological parameters and transmit a signal representing the physiological parameters. For example, the sensing device 115 may be any device configured to measure one or more of the following parameters: intraocular pressure, flow velocity of aqueous humor, blood sugar, and blood biochemicals. Of course, the sensing device 115 may be configured to measure other parameters in addition to or instead of those listed.

In one exemplary approach, the sensing device 115 may be implemented using a micro-electro-mechanical systems (MEMS) device or a nano-electro-mechanical systems (NEMS) device. The sensing device 115 may be disposed in the housing 105 and/or in the fixation element 110. This way, the sensing device 115 may be protected from exposure to, for instance, aqueous humor within the anterior chamber of the patient's eye. Indeed, the characteristics of the housing 105 may be used to increase the accuracy of the sensing device 115. In one exemplary implementation, the housing 105 may be coated with a pressure sensitive coating that helps the sensing device 115 measure, for instance, intraocular pressure. The sensor 100 may include any number of sensing devices 115 at various locations in the housing 105, which aid in balancing the weight of the housing 105.

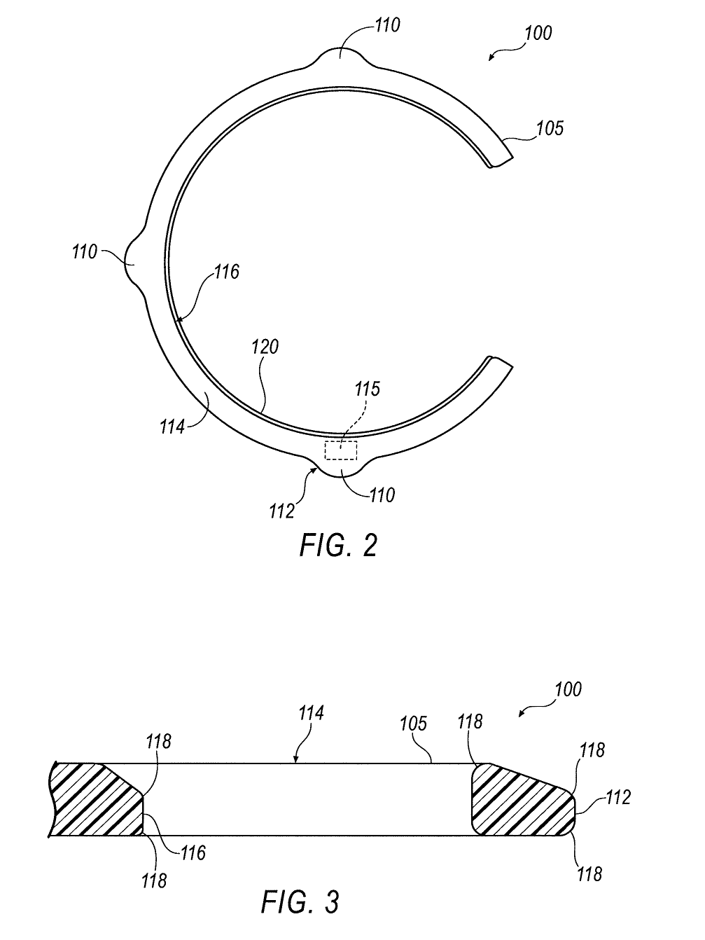

The antenna 120 receives the signals generated by the sensing device 115 and transmits those signals to a receiver located outside the patient's body. The antenna 120 may be disposed on or embedded into the housing 105. For instance, as illustrated in FIGS. 1A-1D, the antenna 120 is embedded within the housing 105. However, as illustrated in FIG. 2, the antenna 120 may alternatively be at least partially disposed on the inside periphery 116 of the housing 105. The sensing device 115 need not have a separate antenna 120. For instance, the housing 105 itself or the patient's body may act as the antenna 120.

FIG. 3 illustrates an exemplary cross-sectional view of a portion of the sensor 100 of FIG. 1A taken along line 3-3 thereof. As shown, portions of the housing 105, such as the portion between the inner periphery 116 and the outer periphery 112, may have a tapered cross-sectional configuration. The cross-sectional shape of the housing 105 may conform to the anterior chamber of the patient's eye. Moreover, the edges 118 of the housing 105 may be rounded for the patient's comfort.

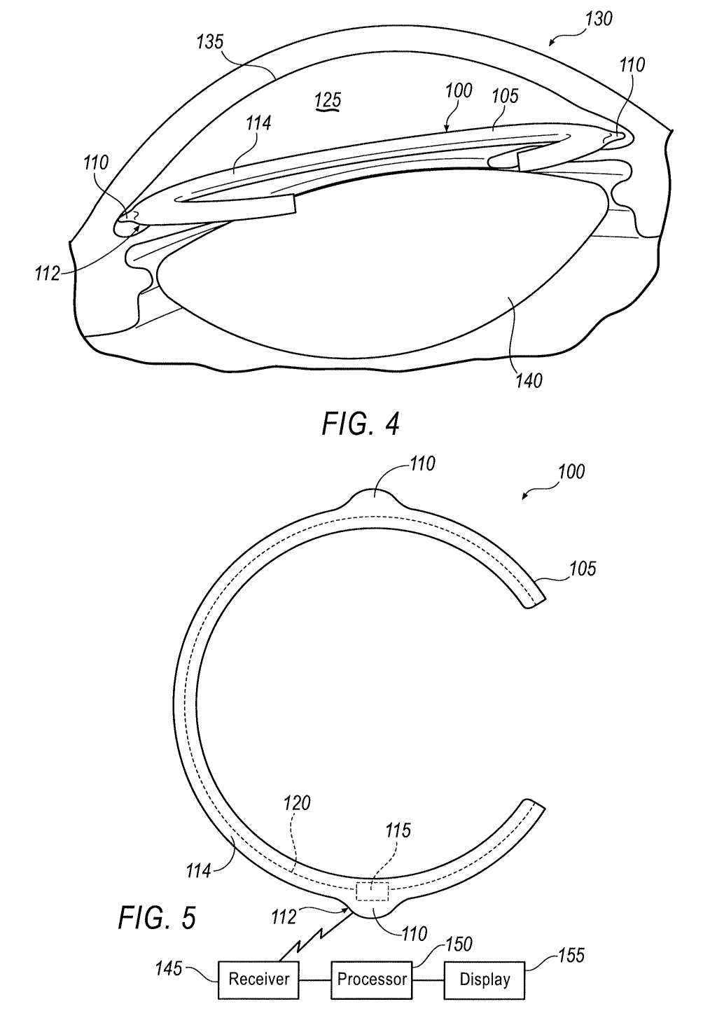

FIG. 4 illustrates an exemplary sensor 100 disposed in the anterior chamber 125 of a patient's eye 130. Specifically, the fixation elements 110 anchor the sensor 100 relative to the patient's corneal endothelium 135, thus limiting movement of the sensor 100 within the anterior chamber 125. In one exemplary approach, the housing 105 may be formed from a flexible material or nanomaterial to allow the sensor 100 to fit within the anterior chamber 125. For instance, when implanting the sensor 100 in the patient's eye, the housing 105 may be distorted to fit within the anterior chamber 125. If distorted, the housing 105 may be biased to return to its previous shape. Also, the shape of the housing 105 allows light to reach the patient's lens 140 because the housing 105 does not, for instance, significantly block light from reaching the patient's lens 140.

Referring now to FIG. 5, a receiver 145 is configured to receive the signal generated by the sensing device 115. In particular, the receiver 145 is in wireless communication with the sensing device 115 via the antenna 120. The receiver 145 may be further configured to output the signals to a processor 150 that can process the signal and determine the sensor 100 readings taken by the sensing device 115. The processor 150 may output the readings to a display device 155 where a graphical representation of the readings may be viewed and interpreted by a clinician. The clinician may use the readings to diagnose and/or treat the patient.

Computing devices, such as the processor 150, generally include computer-executable instructions, where the instructions may be executable by one or more computing devices such as those listed above. Computer-executable instructions may be compiled or interpreted from computer programs created using a variety of well known programming languages and/or technologies, including, without limitation, and either alone or in combination, Java™, C, C++, Visual Basic, Java Script, Perl, etc. In general, a processor (e.g., a microprocessor) receives instructions, e.g., from a memory, a computer-readable medium, etc., and executes these instructions, thereby performing one or more processes, including one or more of the processes described herein. Such instructions and other data may be stored and transmitted using a variety of known computer-readable media.

A computer-readable medium (also referred to as a processor-readable medium) includes any non-transitory (e.g., tangible) medium that participates in providing data (e.g., instructions) that may be read by a computer (e.g., by a processor of a computer). Such a medium may take many forms, including, but not limited to, non-volatile media and volatile media. Non-volatile media may include, for example, optical or magnetic disks and other persistent memory. Volatile media may include, for example, dynamic random access memory (DRAM), which typically constitutes a main memory. Such instructions may be transmitted by one or more transmission media, including coaxial cables, copper wire and fiber optics, including the wires that comprise a system bus coupled to a processor of a computer. Common forms of computer-readable media include, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH-EEPROM, any other memory chip or cartridge, or any other medium from which a computer can read.

In some examples, system elements may be implemented as computer-readable instructions (e.g., software) on one or more computing devices (e.g., servers, personal computers, etc.), stored on computer readable media associated therewith (e.g., disks, memories, etc.). A computer program product may comprise such instructions stored on computer readable media for carrying out the functions described herein.

In operation, the sensor 100 is surgically implanted inside the patient's eye 130. While in the anterior chamber 120, the sensor 100 may measure the pressure of the aqueous humor to determine intraocular pressure using the sensing device 115. Of course, the sensor 100 may be used to measure other physiological parameters as previously discussed. The sensor 100 may transmit the measured intraocular pressure to the receiver 145 using the antenna 120, which transmits the signal representing intraocular pressure to the processor 150. The processor 150 may convert the measured intraocular pressure signal to a signal that graphically represents the intraocular pressure to the display device 155.

With regard to the processes, systems, methods, heuristics, etc. described herein, it should be understood that, although the steps of such processes, etc. have been described as occurring according to a certain ordered sequence, such processes could be practiced with the described steps performed in an order other than the order described herein. It further should be understood that certain steps could be performed simultaneously, that other steps could be added, or that certain steps described herein could be omitted. In other words, the descriptions of processes herein are provided for the purpose of illustrating certain embodiments, and should in no way be construed so as to limit the claimed invention.

Accordingly, it is to be understood that the above description is intended to be illustrative and not restrictive. Many embodiments and applications other than the examples provided would be apparent upon reading the above description. The scope of the invention should be determined, not with reference to the above description, but should instead be determined with reference to the appended claims, along with the full scope of equivalents to which such claims are entitled. It is anticipated and intended that future developments will occur in the technologies discussed herein, and that the disclosed systems and methods will be incorporated into such future embodiments. In sum, it should be understood that the invention is capable of modification and variation.

All terms used in the claims are intended to be given their broadest reasonable constructions and their ordinary meanings as understood by those knowledgeable in the technologies described herein unless an explicit indication to the contrary in made herein. In particular, use of the singular articles such as “a,” “the,” “said,” etc. should be read to recite one or more of the indicated elements unless a claim recites an explicit limitation to the contrary.

1. A sensor comprising:
a housing having a configuration that generally conforms to a curvature of an anterior chamber of a patient's eye; and
a sensing device disposed in the housing and configured to detect physiological parameters and transmit a signal representing the physiological parameters.

2. A sensor as set forth in claim 1, further comprising at least two fixation elements connected to the housing and configured to anchor the housing inside the anterior chamber of the patient's eye.

3. A sensor as set forth in claim 2, wherein at least one of the two fixation elements includes at least two protrusions extending from an outer periphery of the housing.

4. A sensor as set forth in claim 2, wherein at least one of the two fixation elements is integrally formed with the housing.

5. A sensor as set forth in claim 2, wherein at least one of the two fixation elements is integrally formed with the housing and wherein the sensing device is disposed in the fixation element.

6. A sensor as set forth in claim 1, wherein the housing is formed from a flexible material or nanomaterial.

7. A sensor as set forth in claim 1, wherein the housing is coated with a pressure sensitive coating.

8. A sensor as set forth in claim 1, further comprising an antenna in communication with the sensing device.

9. A sensor as set forth in claim 1, wherein a portion of the periphery of the housing has a tapered cross-sectional configuration.

10. A sensor as set forth in claim 1, wherein the sensing device is configured to measure at least one of the following parameters: intraocular pressure, flow velocity of aqueous humor, blood sugar, and blood biochemicals.

11. A sensor as set forth in claim 1, wherein the shape of the housing is configured to allow light to reach the patient's retina and reduce damage to the iris and corneal endothelium.

12. A sensor as set forth in claim 1, wherein the housing has a generally C-shaped configuration.

13. A system comprising:
an implantable sensor having a housing that generally conforms to a curvature of an anterior chamber of a patient's eye and a sensing device configured to detect physiological parameters and transmit a signal representing the physiological parameters;
a receiver configured to receive the signal from the implantable sensor;
a processor configured to receive the signal from the receiver and process the signal; and
a display device configured to display a graphical representation of the signal.

14. A system as set forth in claim 13, further comprising a display device in communication with the receiver and configured to display a graphical representation of the physiological parameter.

15. A system as set forth in claim 14, wherein the implantable sensor includes at least two fixation elements configured to anchor the implantable sensor inside the patient's eye.

16. A system as set forth in claim 15, wherein at least one of the two fixation elements includes at least one protrusion extending from an outer periphery of the housing.

17. A system as set forth in claim 13, wherein the implantable sensor includes an antenna in communication with the sensing device and configured to transmit the physiological parameter to the receiver.

18. A system as set forth in claim 13, wherein the sensing device is configured to measure at least one of the following parameters: intraocular pressure, flow velocity of aqueous humor, blood sugar, and blood biochemicals.

19. A system as set forth in claim 13, wherein the shape of the implantable sensor is configured to limit pupil blockage and corneal endothelium damage.

20. An implantable sensor comprising:
a housing having a substantially C-shaped configuration that generally conforms to a curvature of an anterior chamber of a patient's eye, wherein the shape of the housing is configured to allow light to reach the patient's retina and reduce damage to the corneal endothelium and wherein the housing defines at least two fixation elements configured to anchor the housing inside the patient's eye;
a sensing device embedded in the housing and configured to detect physiological parameters and transmit a signal representing the physiological parameters; and
an antenna in communication with the sensing device.

21. The implantable sensor as set forth in claim 20, wherein the sensing device is configured to measure at least one of the following parameters: intraocular pressure, flow velocity of aqueous humor, blood sugar, and blood biochemicals.
